(19) United States
(12) Patent Application Publication
(10) Pub. No.: US 2022/0064195 A1
(43) Pub. Date: Mar. 3, 2022

(54) SYNTHETIC I2 IMIDAZOLINE RECEPTOR LIGANDS FOR PREVENTION OR TREATMENT OF HUMAN BRAIN DISORDERS

(71) Applicants: Universitat de Barcelona, Barcelona (ES); Universidad del País Vasco, Leioa (ES); Universitat de les Illes Balears, Palma (ES)

(72) Inventors: María Carmen ESCOLANO MIRÓN, Barcelona (ES); Mercè PALLÀS LLIBERIA, Barcelona (ES); Cristian Gaspar GRIÑÁN FERRE, Barcelona (ES); Sònia ABÁS PRADES, Barcelona (ES); Luis-Felipe CALLADO HERNANDO, Leioa (ES); Jesús A. GARCÍA SEVILLA, Palma (ES)

(21) Appl. No.: 17/413,738

(22) PCT Filed: Dec. 19, 2018
(86) PCT No.: PCT/EP2018/085742

§ 371 Date: Jun. 14, 2021

(51) Int. Cl. C07F 9/6561; A61P 25/28

(52) U.S. Cl. CPC C07F 9/6561; A61P 25/28

(57) ABSTRACT

Compounds of formula I, their respective mirror-image enantiomers, and mixtures—preferably racemic—of both enantiomers, wherein R₁ is ethyl or phenyl; R₂ is methyl, phenyl, monosubstituted phenyl, benzyl, or monosubstituted benzyl; R₃ is selected from the group consisting of: (C₁-C₆)-alkyl, (C₁-C₆)-cycloalkyl, —[CH₂]ₙ-phenyl, —[CH₂]ₙ-1-naphtyl, —[CH₂]ₙ-2-naphtyl, and —[CH₂]ₙ-[substituted phenyl]; wherein [substituted phenyl] is a phenyl radical with one, two or three substituents independently selected from: F, Cl, Br, (C₁-C₃)-alkyl, (C₁-C₃)-alkyloxy, phenyl, phenoxy, —CF₃, —OCF₃, nitro, —CN, —CO—(C₁-C₃)-alkyl and benzoyl; and n is an integer between 0 and 4; have a high affinity for imidazoline receptors of the I₂ type, i.e. they are I₂-IR ligands. Consequently they are applicable in the prevention or treatment of brain disorders in animals, including humans, particularly of neurodegenerative disorders, and more particularly of Alzheimer's disease (AD).

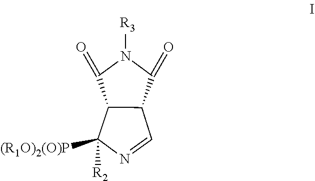

### TECHNICAL FIELD

The present invention relates to the field of compounds with high affinity for imidazoline receptors of the I₂-type (compounds referred to as I₂-IR ligands in the following), which have been associated with prevention or treatment of human brain disorders.

### BACKGROUND ART

In the research in the field of imidazoline receptors much attention was focused on I-type receptors and potential clinical implication such as hypertension. However, the understanding of I₂-IR and its potential functionality had always been elusive until recently. Although there is a lack of consensus regarding the nature of I₂-IR, it seems clear that I₂-IR may represent a group of heterogeneous proteins recognized by I₂-IR ligands such as idazoxan, the ligand mainly used to characterize I₂-IR. I₂-IR are involved in human brain disorders, such as depression and Alzheimer's disease (AD), pain modulation, and inflammation. I₂-IR ligands show antihyperalgesic properties in a murine model of inflammatory and neuropathic pain. In AD brain patiens the density (Bmax) of I₂ imidazoline sites is significantly higher than in control, and neuroprotectant properties have been demonstrated (cf. S. Abas et al., “Neuroprotective Effects of a Structurally New Family of High Affinity Imidazoline I₂ Receptor Ligands”, ACS Chemical Neuroscience 2017, vol. 8, pp. 737-742; and references therein). It is believed that I₂-IR ligands are a new first-in-class of pharmacotherapeutics for the management of brain disorders such as neuropathic pain and inflammation. Selective I₂-IR ligands devoid of □₂-adrenergic receptor (□₂-AR) activity are considered particularly promising as active pharmaceutical ingredients (API), as illustrated in recent reviews (cf. e.g. J.-X. Li, “Imidazoline I₂ receptors: An update”, Pharmacology & Therapeutics 2017. vol. 178, pp. 48-56; J.-X. Li et al., “Imidazoline I₂ receptors: Target for new analgesics?”, European Journal of Pharmacology 2011, vol. 658, pp. 49-56; and references therein), in which it is also shown that most synthetic I₂-IR ligands have an imidazoline moiety. Thus, prior art suggests that it is desirable to provide new I₂-IR ligands useful as API.

### SUMMARY OF INVENTION

An aspect of the present invention relates to the provision of a compound selected from the group consisting of the enantiomer of formula I, its mirror-image enantiomer, and a mixture of both enantiomers, wherein:

R₁ is ethyl or phenyl;
R₂ is methyl, phenyl, benzyl, monosubstituted phenyl, or monosubstituted benzyl, with a substituent being F, Cl, Br, (C₁-C₃)-alkyl, or (C₁-C₃)-alkyloxy; and
R₃ is a radical selected from the group consisting of: (C₁-C₆)-alkyl, (C₁-C₆)-cycloalkyl, —[CH₂]ₙ-phenyl, —[CH₂]ₙ-1-naphtyl, —[CH₂]ₙ-2-naphtyl, and —[CH₂]ₙ-[substituted phenyl];
wherein [substituted phenyl] is a phenyl radical with one, two or three substituents which are radicals independently selected from the group consisting of: F, Cl, Br, (C₁-C₃)-alkyl, (C₁-C₃)-alkyloxy, phenyl, phenoxy, —CF₃, —OCF₃, nitro, —CN, —CO—(C₁-C₃)-alkyl, and benzoyl; and n is an integer between 0 and 4.

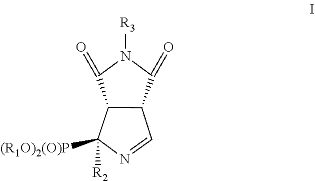

As illustrated by the accompanying experimental results of Table 1, the compounds of the present invention are I₂-IR ligands, and consequently they are applicable in the prevention or treatment of brain disorders. The supplementary experimental results of Tables 2-9 further illustrate their promising potentialities in this respect. For instance, results obtained in cognitive evaluation test in mice indicate that I-06 improves cognitive abilities in a model of aging gated to cognitive decline and early AD and in a murine model of AD. Moreover, molecular and biochemical measures in treated mice hippocampus (an important area for learning and also one of early affected in AD) show a potent and efficient anti-inflammatory effect in both tested models.

Another aspect of the present invention refers to the compounds of the present invention for use as an API. Another aspect of the present invention refers to the compounds of the present invention for use in the prevention or treatment of a brain disorder in an animal, including a human. In a particular embodiment, the brain disorder is a neurodegenerative disorder; and in another particular embodiment, the neurodegenerative disorder is AD. All these aspects of the present invention are related to the use of the compounds of the present invention for the preparation of medicaments for the prevention or treatment of a brain disorder in animals, including humans, particularly neurodegenerative disorders, and more particularly AD. These aspects of the present invention are also related to a method of prevention or treatment of brain disorders, particularly neurodegenerative disorders, and more particularly AD, in animals including humans.

### Preparation Process of Racemic Mixtures Corresponding to Compounds I

Racemic mixtures of compounds I and their respective mirror-image enantioners are prepared by a process involving a diastereoselective [3+2] cycloaddition reaction in conditions analogous to those of a known reaction (cf. C. Arroniz et al., “First diastereoselective [3+2] cycloaddition reaction of diethyl isocyanomethylphosphonate and maleimides”, Org. Biomol. Chem. 2013, vol. 11, pp. 1640-1649), a process which is summarized in the accompanying schemes, and further illustrated in accompanying preparative examples 1-12. The starting materials are either commercially available or prepared as it is here indicated. Diethyl α-methylisocyanomethylphosphonate and diethyl α-benzylisocyanomethylphosphonate are obtained by a known process (cf. J. Rachon et al., “Syntheses with α-metalated isocyanides. XLVIII. Phosphorus analogs of amino acids and peptides. V. Synthesis of 1-aminoalkylphosphonic acids by alkylation of α-metalated diethyl isocyanomethylphosphonate”, Liebigs Ann. Chem. 1981, pp. 709-718). Diethyl α-phenylisocyanomethylphosphonate is obtained by a known process (cf. S. Abas et al., “Easy access to (2-imidazolin-4-yl)phosphonates by a microwave assisted multicomponent reaction”, Tetrahedron 2015, vol. 71, pp. 2872-2881). Diphenyl α-phenylisocyanomethylphosphonate is obtained by a known process (cf. M. Sieńczyk, “Synthesis of isocyanide derivatives of α-aminoalkylphosphonate diphenyl esters”, Tetrahedron Lett. 2006, vol. 47, pp. 4209-4211). Diethyl α-(4-fluorophenyl)-isocyanomethylphosphonate and diethyl α-(4-methoxyphenyl)-isocyanomethylphosphonate are obtained by a known process (cf. S. Sobhani et al., “Molecular Iodine: An efficient catalyst for the one-pot synthesis of primary 1-aminophosphonates”, J. Iran. Chem. Soc. 2010, vol. 7, pp. 227-236). N—R₃ substituted maleimides are commercially available, or they can be prepared by a known process (cf. M. Sortino et al. “Antifungal, cytotoxic and SAR studies of a series of N-alkyl, N-aryl and N-alkylphenyl-1,4-pyrrolediones and related compounds”, Bioorg. Med. Chem. 2011, vol. 19, pp. 2823-2834). Along the description and claims, specific racemic mixtures are referred to by I-#, wherein # is an arbitrary Arabic numeral.

Enantiomers of formula I and their mirror-image enantiomers can be prepared from their respective mixtures by methods known in the art, e.g. by preparative chiral HPLC.

Additional objects, advantages and features of the invention will become apparent to those skilled in the art upon examination of the description or may be learned by practice of the invention. The following examples are provided by way of illustration, and they are not intended to be limiting of the present invention.

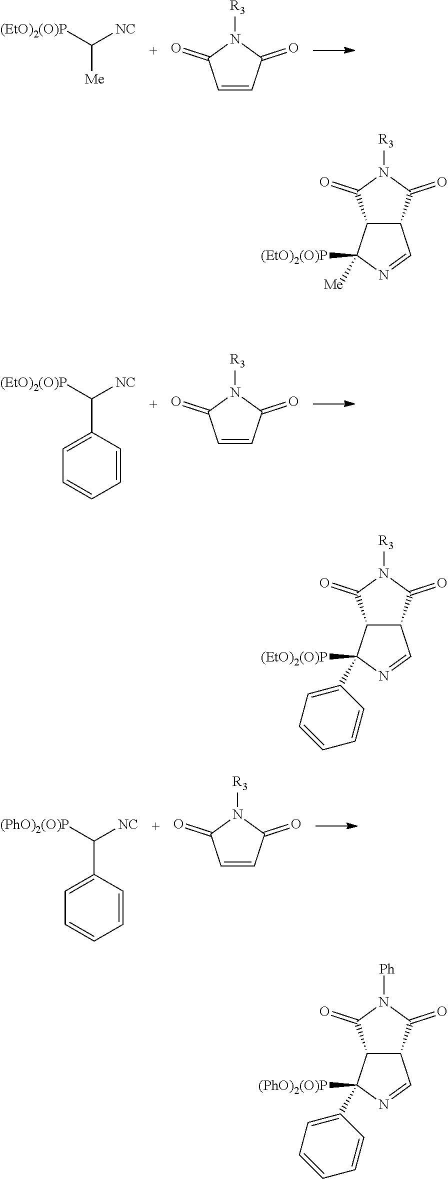

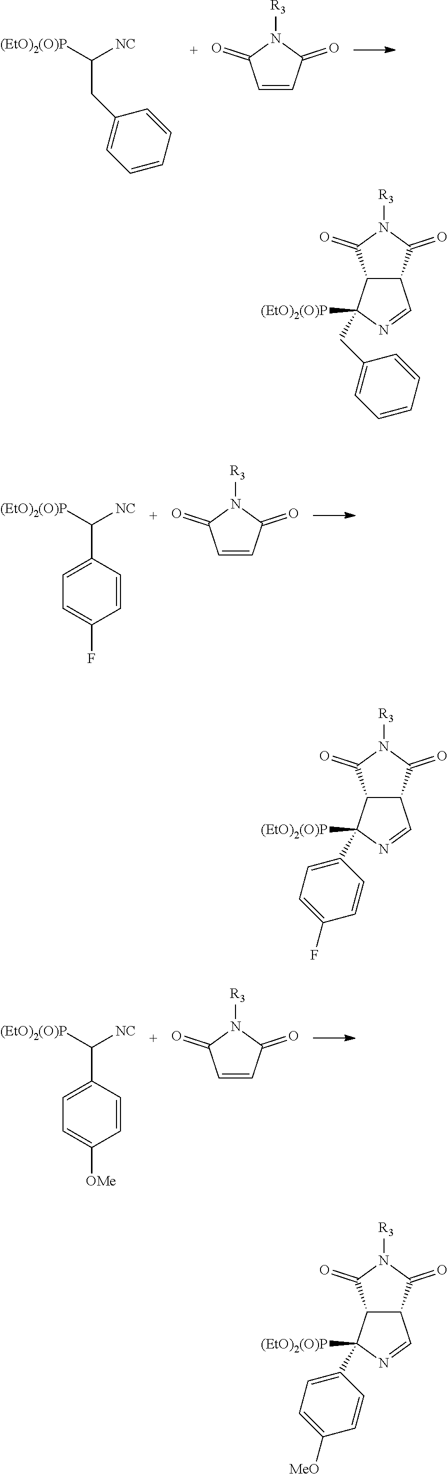

### DESCRIPTION OF EMBODIMENTS

In a particular embodiment the compound of the present invention is a mixture of both enantiomers, and more particularly a racemic mixture of them. In another particular embodiment the substituted phenyl is a phenyl radical with one or two substituents. In another particular embodiment n is an integer between 0 and 2. In another particular embodiment R₃ is selected from the group consisting of: methyl, ethyl, n-propyl, tert-butyl, cyclohexyl, phenyl, 1-naphtyl, 4-methylphenyl, 4-methoxyphenyl, 4-phenoxyphenyl, 3-(trifluoromethyl)phenyl, 4-(trifluoromethyl)phenyl, 4-fluorophenyl, 2-, 3- and 4-chlorophenyl, 4-bromophenyl, 3- and 4-nitrophenyl, 3,4- and 3,5-dichlorophenyl, 3-chloro-4-fluorophenyl, 2-methyl-5-nitrophenyl, (1,1′-biphenyl)-4-yl, benzyl, phenethyl and 4-fluorophenethyl.

Particular embodiments are the following racemic mixtures, prepared for the first time as illustrated in the accompanying examples, as well as their corresponding separated enantiomers:
diethyl (1RS,3aRS,6aRS)-1,5-dimethyl-4,6-dioxo-1,3a,4,5,6,6a-hexahydropyrrolo[3,4-c]pyrrole-1-phosphonate (I-25);
diethyl (1RS,3aRS,6aRS)-1-methyl-4,6-dioxo-5-phenyl-1,3a,4,5,6,6a-hexahydropyrrolo[3,4-c]pyrrole-1-phosphonate (I-31);
diethyl (1RS,3aSR,6aSR)-5-methyl-4,6-dioxo-1-phenyl-1,3a,4,5,6,6a-hexahydropyrrolo[3,4-c]pyrrole-1-phosphonate (I-30);
diethyl (1RS,3aSR,6aSR)-5-cyclohexyl-4,6-dioxo-1-phenyl-1,3a,4,5,6,6a-hexahydropyrrolo[3,4-c]pyrrole-1-phosphonate (I-07);
diethyl (1RS,3aSR,6aSR)-5-benzyl-4,6-dioxo-1-phenyl-1,3a,4,5,6,6a-hexahydropyrrolo[3,4-c]pyrrole-1-phosphonate (I-29);
diethyl (1RS,3aSR,6aSR)-5-(3-nitrophenyl)-4,6-dioxo-1-phenyl-1,3a,4,5,6,6a-hexahydropyrrolo[3,4-c]pyrrole-1-phosphonate (I-28);
diethyl (1RS,3aSR,6aSR)-4,6-dioxo-1,5-diphenyl-1,3a,4,5,6,6a-hexahydropyrrolo[3,4-c]pyrrole-1-phosphonate (I-16);
diethyl (1RS,3aSR,6aSR)-5-(4-methoxyphenyl)-4,6-dioxo-1-phenyl-1,3a,4,5,6,6a-hexahydropyrrolo[3,4-c]pyrrole-1-phosphonate (I-22);
diphenyl (1RS,3aSR,6aSR)-4,6-dioxo-1,5-diphenyl-1,3a,4,5,6,6a-hexahydropyrrolo[3,4-c]pyrrole-1-phosphonate (I-26);
diethyl (1RS,3aSR,6aSR)-4,6-dioxo-5-phenethyl-1-phenyl-1,3a,4,5,6,6a-hexahydropyrrolo[3,4-c]pyrrole-1-phosphonate (I-33); and
diethyl (1RS,3aSR,6aSR)-5-(1,1′-biphenyl)-4-yl-4,6-dioxo-1-phenyl-1,3a,4,5,6,6a-hexahydropyrrolo[3,4-c]pyrrole-1-phosphonate (I-32);
diethyl (1RS,3aSR,6aSR)-4,6-dioxo-5-(4-phenoxyphenyl)-1-phenyl-1,3a,4,5,6,6a-hexahydropyrrolo[3,4-c]pyrrole-1-phosphonate (I-34)
diethyl (1RS,3aSR,6aSR)-5-(4-fluorophenethyl)-4,6-dioxo-1-phenyl-1,3a,4,5,6,6a-hexahydropyrrolo[3,4-c]pyrrole-1-phosphonate (I-36)
diethyl (1RS,3aSR,6aSR)-5-(4-fluorophenyl)-4,6-dioxo-1-phenyl-1,3a,4,5,6,6a-hexahydropyrrolo[3,4-c]pyrrole-1-phosphonate (I-37)
diethyl (1RS,3aSR,6aSR)-4,6-dioxo-1-phenyl-5-[4-(trifluoromethyl)phenyl]-1,3a,4,5,6,6a-hexahydropyrrolo[3,4-c]pyrrole-1-phosphonate (I-38)
diethyl (1RS,3aSR,6aSR)-5-(3,4-dichlorophenyl)-4,6-dioxo-1-phenyl-1,3a,4,5,6,6a-hexahydropyrrolo[3,4-c]pyrrole-1-phosphonate (I-44);
diethyl (1RS,3aSR,6aSR)-4,6-dioxo-1-phenyl-5-(p-tolyl)-1,3a,4,5,6,6a-hexahydropyrrolo[3,4-c]pyrrole-1-phosphonate (I-45);
diethyl (1RS,3aSR,6aSR)-1-benzyl-4,6-dioxo-5-phenyl-1,3a,4,5,6,6a-hexahydropyrrolo[3,4-c]pyrrole-1-phosphonate (I-46);
diethyl (1RS,3aSR,6aSR)-5-(4-bromophenyl)-4,6-dioxo-1-phenyl-1,3a,4,5,6,6a-hexahydropyrrolo[3,4-c]pyrrole-1-phosphonate (I-47);
diethyl (1RS,3aSR,6aSR)-5-(4-chlorophenyl)-4,6-dioxo-1-phenyl-1,3a,4,5,6,6a-hexahydropyrrolo[3,4-c]pyrrole-1-phosphonate (I-49);
diethyl (1RS,3aSR,6aSR)-5-(2-methyl-5-nitrophenyl)-4,6-dioxo-1-phenyl-1,3a,4,5,6,6a-hexahydropyrrolo[3,4-c]pyrrole-1-phosphonate (I-50);
diethyl (1RS,3aSR,6aSR)-4,6-dioxo-1-phenyl-5-propyl-1,3a,4,5,6,6a-hexahydropyrrolo[3,4-c]pyrrole-1-phosphonate (I-51);
diethyl (1RS,3aSR,6aSR)-4,6-dioxo-1-phenyl-5-[3-(trifluoromethyl)phenyl]-1,3a,4,5,6,6a-hexahydropyrrolo[3,4-c]pyrrole-1-phosphonate (I-52);
diethyl (1RS,3aSR,6aSR)-5-(3-chlorophenyl)-4,6-dioxo-1-phenyl-1,3a,4,5,6,6a-hexahydropyrrolo[3,4-c]pyrrole-1-phosphonate (I-53);
diethyl (1RS,3aSR,6aSR)-5-ethyl-4,6-dioxo-1-phenyl-1,3a,4,5,6,6a-hexahydropyrrolo[3,4-c]pyrrole-1-phosphonate (I-54);
diethyl (1RS,3aSR,6aSR)-5-(tert-butyl)-4,6-dioxo-1-phenyl-1,3a,4,5,6,6a-hexahydropyrrolo[3,4-c]pyrrole-1-phosphonate (I-55);
diethyl (1RS,3aSR,6aSR)-5-(naphth-1-yl)-4,6-dioxo-1-phenyl-1,3a,4,5,6,6a-hexahydropyrrolo[3,4-c]pyrrole-1-phosphonate (I-56);
diethyl (1RS,3aSR,6aSR)-1-benzyl-5-cyclohexyl-4,6-dioxo-1,3a,4,5,6,6a-hexahydropyrrolo[3,4-c]pyrrole-1-phosphonate (I-57);
diethyl (1RS,3aSR,6aSR)-5-(3,5-dichlorophenyl)-4,6-dioxo-1-phenyl-1,3a,4,5,6,6a-hexahydropyrrolo[3,4-c]pyrrole-1-phosphonate (I-58);
diethyl (1RS,3aSR,6aSR)-5-(4-nitrophenyl)-4,6-dioxo-1-phenyl-1,3a,4,5,6,6a-hexahydropyrrolo[3,4-c]pyrrole-1-phosphonate (I-59);
diethyl (1RS,3aSR,6aSR)-5-(3-chloro-4-fluorophenyl)-1-(4-fluorophenyl)-4,6-dioxo-1,3a,4,5,6,6a-hexahydropyrrolo[3,4-c]pyrrol-1-phosphonate (I-62);
diethyl (1RS,3aSR,6aSR)-5-(3-chloro-4-fluorophenyl)-1-(4-methoxyphenyl)-4,6-dioxo-1,3a,4,5,6,6a-hexahydropyrrolo[3,4-c]pyrrole-1-phosphonate (I-64);
diethyl (1RS,3aSR,6aSR)-1-(4-fluorophenyl)-4,6-dioxo-5-phenyl-1,3a,4,5,6,6a-hexahydropyrrolo[3,4-c]pyrrole-1-phosphonate (I-65);
diethyl (1RS,3aSR,6aSR)-1-(4-methoxyphenyl)-4,6-dioxo-5-phenyl-1,3a,4,5,6,6a-hexahydropyrrolo[3,4-c]pyrrole-1-phosphonate (I-66);
diethyl (1RS,3aSR,6aSR)-5-cyclohexyl-1-(4-fluorophenyl)-4,6-dioxo-1,3a,4,5,6,6a-hexahydropyrrolo[3,4-c]pyrrole-1-phosphonate (I-67);
diethyl (1RS,3aSR,6aSR)-5-cyclohexyl-1-(4-methoxyphenyl)-4,6-dioxo-1,3a,4,5,6,6a-hexahydropyrrolo[3,4-c]pyrrole-1-phosphonate (I-68);
diethyl (1RS,3aSR,6aSR)-5-(2-chlorophenyl)-4,6-dioxo-1-phenyl-1,3a,4,5,6,6a-hexahydropyrrolo[3,4-c]pyrrole-1-phosphonate (I-69); and
diethyl (1RS,3aSR,6aSR)-5-(3-chloro-4-fluorophenyl)-4,6-dioxo-1-phenyl-1,3a,4,5,6,6a-hexahydropyrrolo[3,4-c]pyrrole-1-phosphonate (I-06), which is the preferred one, used for illustrative purposes in experiments of Tables 3-9.

### General Process for the [3+2] Cycloaddition Reaction to Prepare the Racemic Mixtures Corresponding to Compounds I (Specific Racemic Mixtures are Referred to by I-#, Wherein # is an Arbitrary Arabic Numeral)

Reagents, solvents and starting materials were acquired from commercial sources. The term “concentration” refers to the vacuum evaporation using a Büchi rotavapor. When indicated, the reaction products were purified by “flash” chromatography on silica gel (35-70 μm) with the indicated solvent system. Melting points were measured in a MFB 59510M Gallenkamp instruments. IR spectra were performed in a spectrophotometer Nicolet Avantar 320 FTR-IR or in a Spectrum Two FT-IR Spectrometer, and only noteworthy IR absorptions (cm⁻¹) are listed. Accurate mass analyses were carried out using a LC/MSD-TOF spectrophotometer. Elemental analyses were carried out in a Flash 1112 series Thermofinnigan elemental microanalyzator (A5) to determine C, H, and N.

To a solution of silver acetate (0.06 or 0.10 mmol) and N—R₃ substituted maleimide (1.0 or 1.5 mmol) in acetonitrile, 1.0 mmol of diethyl α-methylisocyanomethylphosphonate, diethyl α-phenylisocyanomethylphosphonate, diphenyl α-phenylisocyanomethyl-phosphonate, diethyl α-(4-fluorophenyl)isocyanomethylphosphonate, diethyl α-(4-methoxyphenyl)-isocyanomethylphosphonate, or diethyl α-benzylisocyanomethyl-phosphonate was added. The reaction mixture was stirred at room temperature overnight, concentrated, and the resulting residue was purified by column chromatography to afford racemic mixtures I-#.

### Example 1. Diethyl (1RS,3aRS,6aRS)-1,5-dimethyl-4,6-dioxo-1,3a,4,5,6,6a-hexahydropyrrolo[3,4-c]pyrrole-1-phosphonate (I-25)

Following the general procedure, AgOAc (20 mg, 0.12 mmol), N-methylmaleimide (222 mg, 2.0 mmol), acetonitrile (15 mL) and diethyl α-methylisocyanomethylphosphonate (384 mg, 2.0 mmol) gave I-25 (239 mg, 40%) as a yellowish oil, after column chromatography (EtOAc). IR (NaCl) 3467, 2982, 1706, 1434, 1380, 1283, 1123, 1050, 1021, 973, 770 cm⁻¹. HRMS C₁₂H₂₀N₂O₅P [M+H]⁺ 303.1105; found, 303.1104.

### Example 2. Diethyl (1RS,3aRS,6aRS)-1-methyl-4,6-dioxo-5-phenyl-1,3a,4,5,6,6a-hexahydropyrrolo[3,4-c]pyrrole-1-phosphonate (I-31)

Following the general procedure, AgOAc (8 mg, 0.05 mmol), N-phenylmaleimide (208 mg, 1.2 mmol), acetonitrile (6 mL) and diethyl α-methylisocyanomethylphosphonate (153 mg, 0.8 mmol) gave I-31 (35 mg, 12%) as a coloured oil, after column chromatography (EtOAc/hexane 8:2 to 9:1). IR (NaCl) 3479, 2984, 1704, 1455, 1389, 1241, 1019, 968 cm⁻¹. HRMS C₁₇H₂₂N₂O₅P [M+H]⁺ 365.1259; found, 365.1261.

### Example 3. Diethyl (1RS,3aSR,6aSR)-5-methyl-4,6-dioxo-1-phenyl-1,3a,4,5,6,6a-hexahydropyrrolo[3,4-c]pyrrole-1-phosphonate (I-30)

Following the general procedure, AgOAc (13 mg, 0.08 mmol), N-methylmaleimide (133 mg, 1.2 mmol), acetonitrile (6 mL) and diethyl α-phenylisocyanomethylphosphonate (202 mg, 0.8 mmol) gave I-30 (184 mg, 64%) as a yellowish oil after column chromatography (EtOAc/hexane 95:5). IR (NaCl) 3472, 2981, 1709, 1432, 1281, 1248, 1051, 967 cm⁻¹. HRMS C₁₇H₂₂N₂O₅P [M+H]⁺ 365.1262; found, 365.1261.

### Example 4. Diethyl (1RS,3aSR,6aSR)-5-cyclohexyl-4,6-dioxo-1-phenyl-1,3a,4,5,6,6a-hexahydropyrrolo[3,4-c]pyrrole-1-phosphonate (I-07)

Following the general procedure, AgOAc (15 mg, 0.09 mmol), N-cyclohexylmaleimide (403 mg, 2.3 mmol), acetonitrile (12 mL) and diethyl α-phenylisocyanomethylphosphonate (380 mg, 1.5 mmol) gave I-07 (494 mg, 76%) as a white solid after column chromatography (EtOAc). M.p. 128-132° C. (EtOAc). IR (NaCl) 3467, 2934, 2858, 1705, 1370, 1249, 1191, 1025, 971, 755 cm⁻¹. HRMS C₂₂H₃₀N₂O₅P [M+H]⁺ 433.1892; found, 433.1887. Anal. Cald. for C₂₂H₂₉N₂O₅P: C, 61.10%; H, 6.76%; N, 6.48%; Found: C, 61.42%; H, 6.81%; N, 6.47%.

### Example 5. Diethyl (1RS,3aSR,6aSR)-5-benzyl-4,6-dioxo-1-phenyl-1,3a,4,5,6,6a-hexahydropyrrolo[3,4-c]pyrrole-1-phosphonate (I-29)

Following the general procedure, AgOAc (4 mg, 0.02 mmol), N-benzylmaleimide (112 mg, 0.6 mmol), acetonitrile (3 mL) and diethyl α-phenylisocyanomethylphosphonate (101 mg, 0.4 mmol) gave I-29 (139 mg, 79%) as a yellowish oil, after column chromatography (EtOAc/hexane 9:1). IR (NaCl) 3472, 3068, 2984, 1778, 1698, 1632, 1495, 1249, 1172, 1021, 750, 615 cm⁻¹. HRMS C₂₃H₂₆N₂O₅P [M+H]⁺ 441.1574; found, 441.1579.

### Example 6. Diethyl (1RS,3aSR,6aSR)-5-(3-nitrophenyl)-4,6-dioxo-1-phenyl-1,3a,4,5,6,6a-hexahydropyrrolo[3,4-c]pyrrole-1-phosphonate (I-28)

Following the general procedure, AgOAc (4.0 mg, 0.02 mmol), N-(3-nitrophenyl)maleimide (131 mg, 0.6 mmol), acetonitrile (3 mL) and diethyl α-phenylisocyanomethylphosphonate (101 mg, 0.4 mmol) gave I-28 (101 mg, 54%) as a white solid, after column chromatography (EtOAc). M.p. 192-195° C. (EtOAc). IR (NaCl) 2984, 1724, 1537, 1351, 1248, 1176, 1050, 971, 758, 674 cm⁻¹. HRMS C₂₂H₂₃N₃O₇P [M+H]⁺ 472.1268; found, 472.1276. Anal. Cald. for C₂₂H₂₂N₃O₇P: C, 56.05%; H, 4.70%; N, 8.91%; found: C, 55.73%; H, 4.74%; N, 8.85%.

### Example 7. Diethyl (1RS,3aSR,6aSR)-4,6-dioxo-1,5-diphenyl-1,3a,4,5,6,6a-hexahydropyrrolo[3,4-c]pyrrole-1-phosphonate (I-16)

Following the general procedure, AgOAc (4 mg, 0.02 mmol), N-phenylmaleimide (104 mg, 0.6 mmol), acetonitrile (3 mL) and diethyl α-phenylisocyanomethylphosphonate (101 mg, 0.4 mmol) gave I-16 (108 mg, 64%) as a white solid after column chromatography (EtOAc). M.p. 158-160° C. (EtOAc). IR (NaCl) 3479, 2969, 1713, 1496, 1390, 1239, 1021, 969 cm⁻¹. HRMS C₂₂H₂₄N₂O₅P [M+H]⁺ 427.1418; found, 427.1417. Anal. Cald. for C₂₂H₂₃N₂O₅P: C, 61.97%; H, 5.44%; N, 6.57%; found: C, 62.18%; H, 5.36%; N, 6.43%.

### Example 8. Diethyl (1RS,3aSR,6aSR)-5-(3-chloro-4-fluorophenyl)-4,6-dioxo-1-phenyl-1,3a,4,5,6,6a-hexahydropyrrolo[3,4-c]pyrrole-1-phosphonate (I-06)

Following the general procedure, AgOAc (8 mg, 0.05 mmol), N-(3-chloro-4-fluorophenyl)maleimide (250 mg, 1.11 mmol), acetonitrile (6 mL) and diethyl α-phenylisocyanomethylphosphonate (187 mg, 0.74 mmol) gave I-06 (189 mg, 54%) as white needles after column chromatography (EtOAc). M.p. 185-186° C. (EtOAc). IR (NaCl) 3437, 2956, 1718, 1499, 1256, 1050, 980 cm⁻¹. HRMS C₂₂H₂₂ClFN₂O₅P [M+H]⁺ 479.0935; found, 479.0933. Anal. Cald. for C₂₂H₂₁ClFN₂O₅P: C, 55.18%; H, 4.42%; N, 5.85%; found: C, 55.40%; H, 4.51%; N, 5.57%.

### Example 9. Diethyl (1RS,3aSR,6aSR)-5-(4-methoxyphenyl)-4,6-dioxo-1-phenyl-1,3a,4,5,6,6a-hexahydropyrrolo[3,4-c]pyrrole-1-phosphonate (I-22)

Following the general procedure, AgOAc (4 mg, 0.02 mmol), N-(4-methoxyphenyl)maleimide (122 mg, 0.6 mmol), acetonitrile (3 mL) and diethyl α-phenylisocyanomethylphosphonate (102 mg, 0.4 mmol) gave I-22 (119 mg, 65%) as a white solid after column chromatography (EtOAc). M.p. 167° C. (EtOAc). IR (NaCl) 3477, 2981, 2930, 1715, 1513, 1384, 1251, 1024, 970, 755 cm⁻¹. HRMS C₂₃H₂₆N₂O₆P [M+H]⁺ 457.1519; found, 457.1523. Anal. Cald. for C₂₃H₂₅N₂O₆P: C, 60.52%; H, 5.52%; N, 6.14%; Found: C, 60.71%; H, 5.75%; N, 5.98%.

### Example 10. Diphenyl (1RS,3aSR,6aSR)-4,6-dioxo-1,5-diphenyl-1,3a,4,5,6,6a-hexahydropyrrolo[3,4-c]pyrrole-1-phosphonate (I-26)

Following the general procedure, AgOAc (2 mg, 0.01 mmol), N-phenylmaleimide (52 mg, 0.3 mmol), acetonitrile (2 mL) and diphenyl α-phenylisocyanomethyl-phosphonate (70 mg, 0.2 mmol) gave I-26 (73 mg, 70%) as a yellow solid after column chromatography (EtOAc/hexane 1:4 to 3:7). M.p. 141-143° C. (EtOAc). IR (NaCl) 3482, 2924, 1718, 1489, 1378, 1271, 1184, 1025, 945 cm⁻¹. HRMS C₃₀H₂₄N₂O₅P [M+H]⁺ 523.1412; found, 523.1417. Anal. Cald. for C₃₀H₂₃N₂O₅P: C, 68.96%; H, 4.44%; N, 5.36%; found: C, 68.79%; H, 4.39%; N, 5.07%.

### Example 11. Diethyl (1RS,3aSR,6aSR)-4,6-dioxo-5-phenethyl-1-phenyl-1,3a,4,5,6,6a-hexahydropyrrolo[3,4-c]pyrrole-1-phosphonate (I-33)

Following the general procedure, AgOAc (7 mg, 0.04 mmol), N-phenethylmaleimide (200 mg, 1.00 mmol) acetonitrile (5 mL) and diethyl α-phenylisocyanomethylphosphonate (170 mg, 0.67 mmol) gave I-33 (110 mg, 36%) as an oil after column chromatography (EtOAc/hexane 1:1). IR (NaCl) 3468, 3027, 2981, 1709, 1627, 1394, 1250, 1162, 1052, 1025, 968, 792, 750 cm⁻¹. HMRS C₂₄H₂₈N₂O₅P [M+H]⁺ 455.1730; found, 455.1731.

### Example 12. Diethyl (1RS,3aSR,6aSR)-5-(1,1′-biphenyl)-4-yl-4,6-dioxo-1-phenyl-1,3a,4,5,6,6a-hexahydropyrrolo[3,4-c]pyrrole-1-phosphonate (I-32)

Following the general procedure, AgOAc (5 mg, 0.03 mmol), N-(p-phenylphenyl)maleimide (200 mg, 0.8 mmol) acetonitrile (4 mL) and diethyl α-phenylisocyanomethylphosphonate (134 mg, 0.53 mmol) gave I-32 (129 mg, 49%) as a oil. M. p. 183-184° C. (EtOAc). IR (NaCl) 3483, 2982, 2928, 1716, 1628, 1487, 1378, 1248, 1182, 1052, 1024, 969, 839, 792 cm⁻¹. HMRS C₂₈H₂₈N₂O₅P [M+H]⁺ 503.1730; found, 503.1727. Anal. Cald. for C₂₈H₂₇N₂O₅P: C, 66.93%; H, 5.42%; N, 5.57%; found: C, 66.97%; H, 5.26%; N, 5.33%.

### Example 13. Diethyl (1RS,3aSR,6aSR)-4,6-dioxo-5-(4-phenoxyphenyl)-1-phenyl-1,3a,4,5,6,6a-hexahydropyrrolo[3,4-c]pyrrole-1-phosphonate (I-34)

Following the general procedure, AgOAc (9 mg, 0.05 mmol), N-(4-phenoxyphenyl)maleimide (371 mg, 1.4 mmol), acetonitrile (7 mL) and diethyl α-phenylisocyanomethylphosphonate (228 mg, 0.9 mmol) gave I-34 (302 mg, 65%) as a white solid, after column chromatography (EtOAc). M.p. 165-167° C. (EtOAc). IR (NaCl) 3488, 3057, 2984, 1783, 1715, 1628, 1488, 1242, 1187, 1024, 700, 578 cm⁻¹. HRMS C₂₈H₂₈N₂O₆P [M+H]⁺ 519.1679; found, 519.1675. Anal. Cald. for C₂₈H₂₇N₂O₆P: C, 64.86%; H, 5.25%; N, 5.40%; found: C, 65.12%; H, 5.26%; N, 5.41%.

### Example 14. Diethyl (1RS,3aSR,6aSR)-5-(4-fluorophenethyl)-4,6-dioxo-1-phenyl-1,3a,4,5,6,6a-hexahydropyrrolo[3,4-c]pyrrole-1-phosphonate (I-36)

Following the general procedure, AgOAc (8 mg, 0.05 mmol), N-(4-fluorophenethyl)maleimide (263 mg, 1.2 mmol), acetonitrile (6 mL) and diethyl α-phenylisocyanomethylphosphonate (202 mg, 0.8 mmol) gave I-36 (201 mg, 53%) as a white solid, after column chromatography (EtOAc). M.p. 94-95° C. (EtOAc). IR (NaCl) 3466, 3050, 2976, 1779, 1702, 1632, 1507, 1257, 1153, 1013, 763, 583 cm⁻¹. HRMS C₂₄H₂₇FN₂O₅P [M+H]; 473.1636; found, 473.1640. Anal. Cald. for C₂₄H₂₆FN₂O₅P: C, 61.01%; H, 5.55%; N, 5.93%; found: C, 61.14%; H, 5.74%; N, 5.83%.

### Example 15. Diethyl (1RS,3aSR,6aSR)-5-(4-fluorophenyl)-4,6-dioxo-1-phenyl-1,3a,4,5,6,6a-hexahydropyrrolo[3,4-c]pyrrole-1-phosphonate (I-37)

Following the general procedure, AgOAc (7 mg, 0.04 mmol), N-(4-fluorophenyl)maleimide (211 mg, 1.1 mmol), acetonitrile (5 mL) and diethyl α-phenylisocyanomethylphosphonate (177 mg, 0.7 mmol) gave I-37 (198 mg, 64%) as a white solid, after column chromatography (EtOAc). M.p. 200-201° C. (EtOAc). IR (NaCl) 3481, 3061, 2980, 1787, 1717, 1511, 1385, 1245, 1017, 969, 700, 583 cm⁻¹. HRMS C₂₂H₂₃FN₂O₅P [M+H]⁺ 445.1323; found, 445.1322. Anal. Cald. for C₂₂H₂₂FN₂O₅P: C, 59.46%; H, 4.99%; N, 6.30%; found: C, 59.73%; H, 5.13%; N, 6.19%.

### Example 16. Diethyl (1RS,3aSR,6aSR)-4,6-dioxo-1-phenyl-5-[4-(trifluoromethyl)phenyl]-1,3a,4,5,6,6a-hexahydropyrrolo[3,4-c]pyrrole-1-phosphonate (I-38)

Following the general procedure, AgOAc (4 mg, 0.03 mmol), N-(4-trifluoromethylphenyl)maleimide (144 mg, 0.6 mmol), acetonitrile (3 mL) and diethyl α-phenylisocyanomethylphosphonate (101 mg, 0.4 mmol) gave I-38 (133 mg, 67%) as a white solid, after column chromatography (EtOAc). M.p. 184-185° C. (EtOAc). IR (NaCl) 3492, 3050, 2984, 1723, 1616, 1378, 1326, 1249, 1170, 1067, 758, 580 cm⁻¹. HRMS C₂₃H₂₃F₃N₂O₅P [M+H]⁺ 495.1291; found, 495.1288. Anal. Cald. for C₂₃H₂₂F₃N₂O₅P: C, 55.88%; H, 4.49%; N, 5.67%; found: C, 56.04%; H, 4.71%; N, 5.56%.

### Example 17. Diethyl (1RS,3aSR,6aSR)-5-(3,4-dichlorophenyl)-4,6-dioxo-1-phenyl-1,3a,4,5,6,6a-hexahydropyrrolo[3,4-c]pyrrole-1-phosphonate (I-44)

Following the general procedure, AgOAc (8 mg, 0.05 mmol), N-(3-chloro-4-chlorophenyl)maleimide (288 mg, 1.2 mmol), acetonitrile (6 mL) and diethyl α-phenylisocyanomethylphosphonate (202 mg, 0.8 mmol) gave I-44 (210 mg, 53%) as a white solid, after column chromatography (EtOAc/hexane 1:1). M.p. 172-174° C. (EtOAc). IR (ATR) 3480, 3075, 2957, 1790, 1716, 1464, 1251, 1187, 1058, 1024, 737, 579 cm⁻¹. HRMS C₂₂H₂₂Cl₂N₂O₅P [M+H]⁺ 495.0638; found, 495.0637. Anal. Cald. for C₂₂H₂₁Cl₂N₂O₅P: C, 53.35%; H, 4.27%; N, 5.66%; found: C, 53.42%; H, 4.30%; N, 5.48%.

### Example 18. Diethyl (1RS,3aSR,6aSR)-4,6-dioxo-1-phenyl-5-(p-tolyl)-1,3a,4,5,6,6a-hexahydropyrrolo[3,4-c]pyrrole-1-phosphonate (I-45)

Following the general procedure, AgOAc (6 mg, 0.04 mmol), N-(4-methylphenyl)maleimide (153 mg, 0.9 mmol), acetonitrile (4.5 mL) and diethyl α-phenylisocyanomethylphosphonate (168 mg, 0.6 mmol) gave I-45 (199 mg, 75%) as a white solid, after column chromatography (EtOAc/hexane 3:2 to 9:1). M.p. 156-158° C. (EtOAc). IR (ATR) 3476, 2936, 2863, 1711, 1632, 1520, 1368, 1240, 1181, 1025, 971, 740, 583 cm⁻¹. HRMS C₂₃H₂₆N₂O₅P [M+H]⁺ 441.1574; found, 441.1572. Anal. Cald. for C₂₃H₂₅N₂O₅P: C, 62.72%; H, 5.72%; N, 6.36%; found: C, 62.87%; H, 5.82%; N, 6.18%.

### Example 19. Diethyl (1RS,3aSR,6aSR)-1-benzyl-4,6-dioxo-5-phenyl-1,3a,4,5,6,6a-hexahydropyrrolo[3,4-c]pyrrole-1-phosphonate (I-46)

Following the general procedure, AgOAc (8 mg, 0.05 mmol), N-phenylmaleimide (139 mg, 0.8 mmol), acetonitrile (6 mL) and diethyl α-benzylisocyanomethylphosphonate (213 mg, 0.8 mmol) gave I-46 (32 mg, 9%) as a yellowish oil, after column chromatography (EtOAc/hexane 1:1). IR (ATR) 3738, 2926, 2843, 1730, 1492, 1385, 1220, 1181, 1059, 1020, 782, 700 cm⁻¹. HRMS C₂₃H₂₆N₂O₅P [M+H]⁺ 441.1574; found, 441.1580.

### Example 20. Diethyl (1RS,3aSR,6aSR)-5-(4-bromophenyl)-4,6-dioxo-1-phenyl-1,3a,4,5,6,6a-hexahydropyrrolo[3,4-c]pyrrole-1-phosphonate (I-47)

Following the general procedure, AgOAc (7 mg, 0.04 mmol), N-(4-bromophenyl)maleimide (275 mg, 1.1 mmol), acetonitrile (5 mL) and diethyl α-phenylisocyanomethylphosphonate (177 mg, 0.7 mmol) gave I-47 (181 mg, 51%) as a white solid, after column chromatography (EtOAc/hexane 1:1). M.p. 180-182° C. (EtOAc). IR (ATR) 3478, 2918, 2845, 1797, 1714, 1480, 1387, 1236, 1187, 1158, 1022, 973, 744, 578 cm⁻¹. HRMS C₂₂H₂₃BrN₂O₅P [M+H]⁺ 505.0522; found, 505.0522.

### Example 21. Diethyl (1RS,3aSR,6aSR)-5-(4-chlorophenyl)-4,6-dioxo-1-phenyl-1,3a,4,5,6,6a-hexahydropyrrolo[3,4-c]pyrrole-1-phosphonate (I-49)

Following the general procedure, AgOAc (5 mg, 0.03 mmol), N-(4-chlorophenyl)maleimide (150 mg, 0.7 mmol), acetonitrile (4 mL) and diethyl α-phenylisocyanomethylphosphonate (118 mg, 0.5 mmol) gave I-49 (136 mg, 59%) as a white solid, after column chromatography (EtOAc). M.p. 211-212° C. (EtOAc). IR (ATR) 3078, 2981, 2923, 2855, 1713, 1499, 1377, 1187, 1022, 773, 583 cm⁻¹. HRMS C₂₂H₂₃ClN₂O₅P [M+H]⁺ 461.1028; found, 461.1026. Anal. Cald. for C₂₂H₂₂ClN₂O₅P: C, 57.34%; H, 4.81%; N, 6.08%; found: C, 57.71%; H, 4.92%; N, 5.96%.

### Example 22. Diethyl (1RS,3aSR,6aSR)-5-(2-methyl-5-nitrophenyl)-4,6-dioxo-1-phenyl-1,3a,4,5,6,6a-hexahydropyrrolo[3,4-c]pyrrole-1-phosphonate (I-50)

Following the general procedure, AgOAc (5 mg, 0.03 mmol), N-(2-methyl-5-nitrophenyl)maleimide (138 mg, 0.6 mmol), acetonitrile (3 mL) and diethyl α-phenylisocyanomethylphosphonate (101 mg, 0.4 mmol) gave I-50 (111 mg, 57%) as a white solid, after column chromatography (EtOAc). M.p. 196-198° C. (EtOAc). IR (ATR) 3493, 3079, 2947, 2845, 1724, 1519, 1343, 1192, 1017, 739, 578 cm⁻¹. HRMS C₂₃H₂₅N₃O₇P [M+H]⁺ 486.1425; found, 486.1424. Anal. Cald. for C₂₃H₂₄N₃O₇P: C, 56.91%; H, 4.98%; N, 8.66%; found: C. 57.33%; H, 5.11%; N, 8.59%.

### Example 23. Diethyl (1RS,3aSR,6aSR)-4,6-dioxo-1-phenyl-5-propyl-1,3a,4,5,6,6a-hexahydropyrrolo[3,4-c]pyrrole-1-phosphonate (I-51)

Following the general procedure, AgOAc (8 mg, 0.05 mmol), N-propylmaleimide (200 mg, 1.4 mmol), acetonitrile (7 mL) and diethyl α-phenylisocyanomethylphosphonate (228 mg, 0.9 mmol) gave I-51 (314 mg, 89%) as a yellowish oil, after column chromatography (EtOAc/hexane 1:1 to 3:2). IR (ATR) 3464, 2976, 2928, 1719, 1631, 1451, 1402, 1202, 1056, 1027, 963, 705, 583 cm⁻¹. HRMS C₁₉H₂₆N₂O₅P [M+H]⁺ 393.1574; found, 393.1572.

### Example 24. Diethyl (1RS,3aSR,6aSR)-4,6-dioxo-1-phenyl-5-[3-(trifluoromethyl)phenyl]-1,3a,4,5,6,6a-hexahydropyrrolo[3,4-c]pyrrole-1-phosphonate (I-52)

Following the general procedure, AgOAc (10 mg, 0.06 mmol), N-(3-trifluoromethylphenyl)maleimide (362 mg, 1.5 mmol), acetonitrile (8 mL) and diethyl α-phenylisocyanomethylphosphonate (253 mg, 1.0 mmol) gave I-52 (218 mg, 44%) as a white solid, after column chromatography (EtOAc). M.p. 179-180° C. (EtOAc). IR (ATR) 3483, 3084, 2957, 2036, 1719, 1446, 1382, 1329, 1168, 1027, 978, 739, 573 cm⁻¹. HRMS C₂₃H₂₃F₃N₂O₅P [M+H]⁺ 495.1291; found, 495.1287. Anal. Cald. for C₂₃H₂₂F₃N₂O₅P: C, 55.88%; H, 4.49%; N, 5.67%; found: C, 56.00%; H, 4.63%; N, 5.46%.

### Example 25. Diethyl (1RS,3aSR,6aSR)-5-(3-chlorophenyl)-4,6-dioxo-1-phenyl-1,3a,4,5,6,6a-hexahydropyrrolo[3,4-c]pyrrole-1-phosphonate (I-53)

Following the general procedure, AgOAc (8 mg, 0.05 mmol), N-(3-chlorolphenyl)maleimide (250 mg, 1.2 mmol), acetonitrile (6 mL) and diethyl α-phenylisocyanomethylphosphonate (203 mg, 0.8 mmol) gave I-53 (167 mg, 45%) as a white solid, after column chromatography (EtOAc). M.p. 186-187° C. (EtOAc). IR (ATR) 3488, 3084, 2962, 2928, 1709, 1587, 1475, 1382, 1241, 1183, 1051, 1022, 948, 705 cm⁻¹. HRMS C₂₂H₂₃ClN₂O₅P [M+H]⁺ 461.1028; found, 461.1029. Anal. Cald. for C₂₂H₂₂ClN₂O₅P: C, 57.34%; H, 4.81%; N, 6.08%; found: C, 57.70%; H, 4.96%; N, 5.59%.

### Example 26. Diethyl (1RS,3aSR,6aSR)-5-ethyl-4,6-dioxo-1-phenyl-1,3a,4,5,6,6a-hexahydropyrrolo[3,4-c]pyrrole-1-phosphonate (I-54)

Following the general procedure, AgOAc (11 mg, 0.07 mmol), N-ethylmaleimide (213 mg, 1.7 mmol), acetonitrile (8 mL) and diethyl α-phenylisocyanomethylphosphonate (279 mg, 1.1 mmol) gave I-54 (130 mg, 31%) as a yellowish oil, after column chromatography (EtOAc). IR (ATR) 3476, 2981, 2929, 1780, 1698, 1626, 1396, 1251, 1055, 1026, 968, 766 cm⁻¹. HRMS C₁₈H₂₄N₂O₅P [M+H]⁺ 379.1417; found, 379.1418.

### Example 27. Diethyl (1RS,3aSR,6aSR)-5-(tert-butyl)-4,6-dioxo-1-phenyl-1,3a,4,5,6,6a-hexahydropyrrolo[3,4-c]pyrrole-1-phosphonate (I-55)

Following the general procedure, AgOAc (9 mg, 0.05 mmol), N-tert-butylmaleimide (215 mg, 1.4 mmol), acetonitrile (7 mL) and diethyl α-phenylisocyanomethylphosphonate (229 mg, 0.9 mmol) gave I-55 (202 mg, 55%) as a yellowish oil, after column chromatography (EtOAc). IR (ATR) 3454, 2981, 2923, 1777, 1709, 1348, 1265, 1241, 1173, 1061, 973, 744, 710, 588 cm⁻¹. HRMS C₂₀H₂₈N₂O₅P [M+H]⁺ 407.1730; found, 407.1733.

### Example 28. Diethyl (1RS,3aSR,6aSR)-5-(naphth-1-yl)-4,6-dioxo-1-phenyl-1,3a,4,5,6,6a-hexahydropyrrolo[3,4-c]pyrrole-1-phosphonate (I-56)

Following the general procedure, AgOAc (3 mg, 0.02 mmol), N-(naphth-1-yl)maleimide (100 mg, 0.5 mmol), acetonitrile (2 mL) and diethyl α-phenylisocyanomethylphosphonate (76 mg, 0.3 mmol) gave I-56 (70 mg, 49%) as a white solid, after column chromatography (EtOAc/hexane 1:1 to 9:1). M.p. 197-198° C. (EtOAc). IR (ATR) 3480, 2927, 2853, 1716, 1598, 1446, 1397, 1358, 1240, 1177, 1039, 1025, 961, 775, 706, 583 cm⁻¹. HRMS C₂₆H₂₆N₂O₅P [M+H]⁺ 477.1574; found, 477.1571. Anal. Cald. For C₂₆H₂₅N₂O₅P: C, 65.54%; H, 5.29%; N, 5.88%; found: C, 65.34%; H, 5.12%; N, 5.65%.

### Example 29. Diethyl (1RS,3aSR,6aSR)-1-benzyl-5-cyclohexyl-4,6-dioxo-1,3a,4,5,6,6a-hexahydropyrrolo[3,4-c]pyrrole-1-phosphonate (I-57)

Following the general procedure, AgOAc (7 mg, 0.04 mmol), N-cyclohexylmaleimide (108 mg, 0.6 mmol), acetonitrile (5 mL) and diethyl α-benzylisocyanomethylphosphonate (161 mg, 0.6 mmol) gave I-57 (33 mg, 12%) as a yellowish oil, after column chromatography (EtOAc/hexane 7:3). IR (ATR) 3052, 2981, 2856, 1703, 1626, 1367, 1247, 1055, 1021, 973, 752, 704, 593 cm⁻¹. HRMS C₂₃H₃₂N₂O₅P [M+H]⁺ 447.2043; found, 447.2047.

### Example 30. Diethyl (1RS,3aSR,6aSR)-5-(3,5-dichlorophenyl)-4,6-dioxo-1-phenyl-1,3a,4,5,6,6a-hexahydropyrrolo[3,4-c]pyrrole-1-phosphonate (I-58)

Following the general procedure, AgOAc (12 mg, 0.07 mmol), N-(3,5-dichlorophenyl)maleimide (267 mg, 1.1 mmol), acetonitrile (5 mL) and diethyl α-phenylisocyanomethylphosphonate (177 mg, 0.7 mmol) gave I-58 (190 mg, 55%) as a white solid, after column chromatography (EtOAc/hexane 8:2). M.p. 207-209° C. (EtOAc). IR (ATR) 3483, 3079, 2952, 2928, 1719, 1573, 1441, 1373, 1226, 1183, 1027, 973, 763, 734, 727, 588 cm⁻¹. HRMS C₂₂H₂₂Cl₂N₂O₅P [M+H]⁺ 495.0638; found, 495.0638. Anal. Cald. for C₂₂H₂₁Cl₂N₂O₅P: C, 53.35%; H, 4.27%; N, 5.66%; found: C, 53.69%; H, 4.35%; N, 5.42%.

### Example 31. Diethyl (1RS,3aSR,6aSR)-5-(4-nitrophenyl)-4,6-dioxo-1-phenyl-1,3a,4,5,6,6a-hexahydropyrrolo[3,4-c]pyrrole-1-phosphonate (I-59)

Following the general procedure, AgOAc (13 mg, 0.08 mmol), N-(4-nitrophenyl)maleimide (261 mg, 1.2 mmol), acetonitrile (6 mL) and diethyl α-phenylisocyanomethylphosphonate (203 mg, 0.8 mmol) gave I-59 (154 mg, 40%) as a beige solid, after column chromatography (EtOAc/hexane 7:3). M.p. 133-135° C. (EtOAc). IR (ATR) 3259, 2991, 2928, 1719, 1529, 1494, 1387, 1246, 1187, 1051, 1022, 973, 700, 573 cm⁻¹. HRMS C₂₂H₂₃N₃O₇P [M+H]⁺ 472.1268; found, 472.1271.

### Example 32. Diethyl (1RS,3aSR,6aSR)-5-(3-chloro-4-fluorophenyl)-1-(4-fluorophenyl)-4,6-dioxo-1,3a,4,5,6,6a-hexahydropyrrolo[3,4-c]pyrrole-1-phosphonate (I-62)

Following the general procedure, AgOAc (7 mg, 0.04 mmol), N-(3-chloro-4-fluorophenyl)maleimide (135 mg, 0.6 mmol), acetonitrile (3 mL) and diethyl α-(4-fluorophenyl)isocyanomethylphosphonate (108 mg, 0.4 mmol) gave I-62 (124 mg, 62%) as a white solid, after column chromatography (EtOAc). M.p. 179-181° C. (EtOAc). IR (ATR) 3483, 2962, 2903, 1719, 1504, 1236, 1051, 1012, 978, 739, 593 cm⁻¹. HRMS C₂₂H₂₁ClF₂N₂O₅P [M+H]⁺ 497.0839; found, 497.0840. Anal. Cald. for C₂₂H₂₀ClF₂N₂O₅P: C, 53.19%; H, 4.06%; N, 5.64%; found: C, 53.45%; H, 4.24%; N, 5.46%.

### Example 33. Diethyl (1RS,3aSR,6aSR)-5-(3-chloro-4-fluorophenyl)-1-(4-methoxyphenyl)-4,6-dioxo-1,3a,4,5,6,6a-hexahydropyrrolo[3,4-c]pyrrole-1-phosphonate (I-64)

Following the general procedure, AgOAc (8 mg, 0.05 mmol), N-(3-chloro-4-fluorophenyl)maleimide (181 mg, 0.8 mmol), acetonitrile (4 mL) and diethyl α-(4-methoxyphenyl)isocyanomethylphosphonate (142 mg, 0.5 mmol) gave I-64 (170 mg, 67%) as a white solid, after column chromatography (EtOAc). M.p. 227-228° C. (EtOAc). IR (ATR) 3481, 2986, 2905, 1771, 1718, 1612, 1497, 1386, 1237, 1184, 1026, 968, 752, 656 cm⁻¹. HRMS C₂₃H₂₄ClFN₂O₆P [M+H]⁺ 509.1039; found, 509.1037. Anal. Cald. for C₂₃H₂₃ClFN₂O₆P: C, 54.29%; H, 4.56%; N, 5.51%; found: C, 54.66%; H, 4.63%; N, 5.36%.

### Example 34. Diethyl (1RS,3aSR,6aSR)-1-(4-fluorophenyl)-4,6-dioxo-5-phenyl-1,3a,4,5,6,6a-hexahydropyrrolo[3,4-c]pyrrole-1-phosphonate (I-65)

Following the general procedure, AgOAc (10 mg, 0.06 mmol), N-phenylmaleimide (156 mg, 0.9 mmol), acetonitrile (4 mL) and diethyl α-(4-fluorophenyl)isocyanomethyl-phosphonate (164 mg, 0.6 mmol) gave I-65 (159 mg, 60%) as a white solid, after column chromatography (EtOAc/hexane 4:1). M.p. 191-193° C. (EtOAc). IR (ATR) 3491, 2991, 2909, 1775, 1718, 1598, 1506, 1377, 1242, 1189, 1016, 982, 742, 598 cm⁻¹. HRMS C₂₂H₂₃FN₂O₅P [M+H]⁺ 445.1323; found, 445.1324.

### Example 35. Diethyl (1RS,3aSR,6aSR)-1-(4-methoxyphenyl)-4,6-dioxo-5-phenyl-1,3a,4,5,6,6a-hexahydropyrrolo[3,4-c]pyrrole-1-phosphonate (I-66)

Following the general procedure, AgOAc (8 mg, 0.05 mmol), N-phenylmaleimide (138 mg, 0.8 mmol), acetonitrile (4 mL) and diethyl α-(4-methoxyphenyl)isocyanomethyl-phosphonate (142 mg, 0.5 mmol) gave I-66 (155 mg, 69%) as a white solid, after column chromatography (EtOAc/hexane 4:1). M.p. 184-186° C. (EtOAc). IR (ATR) 3481, 2986, 2914, 1785, 1713, 1607, 1511, 1386, 1247, 1189, 1021, 737, 694, 598 cm⁻¹. HRMS C₂₃H₂₆N₂O₆P [M+H]⁺ 457.1523; found, 457.1520. Anal. Cald. for C₂₃H₂₅N₂O₆P: C, 60.52%; H, 5.52%; N, 6.14%; found: C, 60.85%; H, 5.51%; N, 5.94%.

### Example 36. Diethyl (1RS,3aSR,6aSR)-5-cyclohexyl-1-(4-fluorophenyl)-4,6-dioxo-1,3a,4,5,6,6a-hexahydropyrrolo[3,4-c]pyrrole-1-phosphonate (I-67)

Following the general procedure, AgOAc (10 mg, 0.06 mmol), N-cyclohexylmaleimide (167 mg, 0.9 mmol), acetonitrile (5 mL) and diethyl α-(4-fluorophenyl)isocyanomethyl-phosphonate (164 mg, 0.6 mmol) gave I-67 (174 mg, 64%) as a beige solid, after column chromatography (EtOAc). M.p. 124-126° C. (EtOAc). IR (ATR) 3462, 2929, 2856, 1703, 1626, 1511, 1377, 1247, 1204, 1055, 1016, 963, 761, 593 cm⁻¹. HRMS C₂₂H₂₉FN₂O₅P [M+H]⁺ 451.1793; found, 451.1793.

### Example 37. Diethyl (1RS,3aSR,6aSR)-5-cyclohexyl-1-(4-methoxyphenyl)-4,6-dioxo-1,3a,4,5,6,6a-hexahydropyrrolo[3,4-c]pyrrole-1-phosphonate (I-68)

Following the general procedure, AgOAc (8 mg, 0.05 mmol), N-cyclohexylmaleimide (143 mg, 0.8 mmol), acetonitrile (4 mL) and diethyl α-(4-methoxyphenyl)isocyanomethyl-phosphonate (142 mg, 0.5 mmol) gave I-68 (164 mg, 71%) as a yellowish oil, after column chromatography (EtOAc). IR (ATR) 3462, 2938, 2852, 1698, 1511, 1247, 1372, 1184, 1016, 761, 579 cm⁻¹. HRMS C₂₃H₃₂N₂O₆P [M+H]⁺ 463.1992; found, 463.1993.

### Example 38. Diethyl (1RS,3aSR,6aSR)-5-(2-chlorophenyl)-4,6-dioxo-1-phenyl-1,3a,4,5,6,6a-hexahydropyrrolo[3,4-c]pyrrole-1-phosphonate (I-69)

Following the general procedure, AgOAc (6 mg, 0.04 mmol), N-(2-chlorophenyl)maleimide (180 mg, 0.9 mmol), acetonitrile (5 mL) and diethyl α-phenylisocyanomethylphosphonate (152 mg, 0.6 mmol) gave I-69 (200 mg, 72%) as a white solid, after column chromatography (EtOAc/hexane 7:3). M.p. 172-174° C. (EtOAc). IR (ATR) 3496, 2981, 2866, 1790, 1718, 1482, 1386, 1237, 1194, 1045, 1021, 973, 757, 579 cm⁻¹. HRMS C₂₂H₂₃ClN₂O₅P [M+H]⁺ 461.1028; found, 461.1025. Anal. Cald. for C₂₂H₂₂ClN₂O₅P: C, 57.34%; H, 4.81%; N, 6.08%; found: C, 57.18%; H, 4.86%; N, 5.88%.

Activity Upon I₂ imidazoline receptors

The pharmacological activity of the prepared compounds was evaluated through competition binding studies against the selective I₂-IR radioligand [³H]-2-BFI or the selective α₂-AR radioligand [³H]RX821002. The studies were performed in membranes from post-mortem human frontal cortex, a brain area that shows an important density of I₂-IRs and α₂-AR. The inhibition constant (Kᵢ) for each compound was obtained and is expressed as the corresponding pKᵢ (Table 1). Idazoxan, a compound with well-established affinity for I₂-IR (pKᵢ=7.27±0.07) and α₂-AR (pKᵢ=7.51±0.07) was used as a reference. The selectivity between receptors was expressed by the I₂/α₂ index. This index is calculated as the antilogarithm of the difference between pKᵢ values for I₂-IR and pKᵢ values for α₂-AR (see Table 1).

TABLE 1
I₂-IR and α₂-AR receptor binding affinities (pKᵢ)

| | | [³H]-2-BFI | [³H]-RX821002 | Selectivity |
|---|---|---|---|---|
| Compound | | I₂ | α₂ | I₂/α₂ |
| Idazoxan | 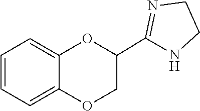 [SMILES: c1ccc2c(c1)OCC(C1=NCCN1)O2] | 7.41 ± 0.63 | 8.35 ± 0.16 | — |
| I-06 | 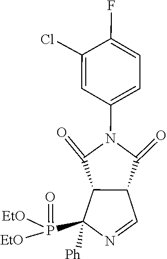 [SMILES: CCOP(=O)(OCC)[C@]1(c2ccccc2)N=C[C@H]2C(=O)N(c3ccc(F)c(Cl)c3)C(=O)[C@H]21] | 8.56 ± 0.32 | 6.27 ± 0.56 | 195 |
| I-07 | 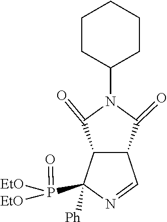 [SMILES: CCOP(=O)(OCC)[C@]1(c2ccccc2)N=C[C@H]2C(=O)N(C3CCCCC3)C(=O)[C@H]21] | 9.74 ± 0.29 | 9.01 ± 0.49 | 5 |
| I-16 | 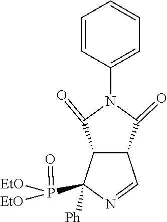 [SMILES: CCOP(=O)(OCC)[C@]1(c2ccccc2)N=C[C@H]2C(=O)N(c3ccccc3)C(=O)[C@H]21] | 10.28 ± 0.37 | 10.38 ± 0.22 | 1 |
| I-22 | 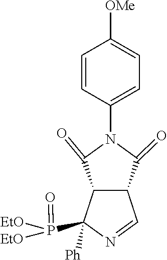 [SMILES: CCOP(=O)(OCC)[C@]1(c2ccccc2)N=C[C@H]2C(=O)N(c3ccc(OC)cc3)C(=O)[C@H]21] | 6.65 ± 1.27 | 4.59 ± 0.22 | 115 |
| I-25 | 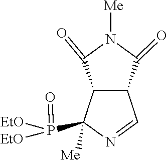 [SMILES: CCOP(=O)(OCC)[C@]1(C)N=C[C@H]2C(=O)N(C)C(=O)[C@H]21] | 8.57 ± 0.39 | 7.29 ± 2.47 | 19 |
| I-26 | 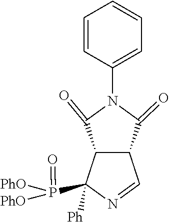 [SMILES: O=C1[C@@H]2[C@@H](C=N[C@@]2(c2ccccc2)P(=O)(Oc2ccccc2)Oc2ccccc2)C(=O)N1c1ccccc1] | 5.86 ± 1.22 | 11.64 ± 0.45 | — |
| I-28 | 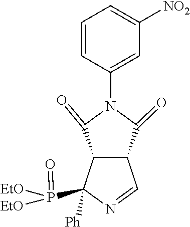 [SMILES: CCOP(=O)(OCC)[C@]1(c2ccccc2)N=C[C@H]2C(=O)N(c3cccc([N+](=O)[O-])c3)C(=O)[C@H]21] | 6.81 ± 0.27 | 10.18 ± 0.41 | |
| I-29 | 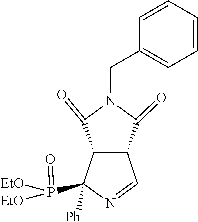 [SMILES: CCOP(=O)(OCC)[C@]1(c2ccccc2)N=C[C@H]2C(=O)N(Cc3ccccc3)C(=O)[C@H]21] | 5.26 ± 0.22 | 8.11 ± 0.28 | |
| I-30 | 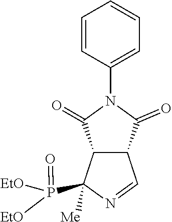 [SMILES: CCOP(=O)(OCC)[C@]1(C)N=C[C@H]2C(=O)N(c3ccccc3)C(=O)[C@H]21] | 7.97 ± 0.55 | 5.93 ± 0.41 | 110 |
| I-32 | 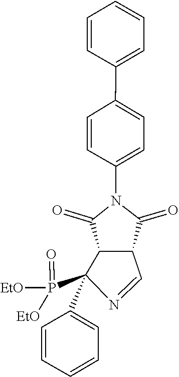 [SMILES: CCOP(=O)(OCC)[C@]1(c2ccccc2)N=C[C@H]2C(=O)N(c3ccc(-c4ccccc4)cc3)C(=O)[C@H]21] | 7.90 ± 0.46 | 5.12 ± 0.14 | 602 |
| I-33 | 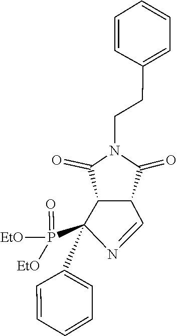 [SMILES: CCOP(=O)(OCC)[C@]1(c2ccccc2)N=C[C@H]2C(=O)N(CCc3ccccc3)C(=O)[C@H]21] | 5.85 ± 0.18 | 3.59 ± 0.25 | 182 |
| I-34 | 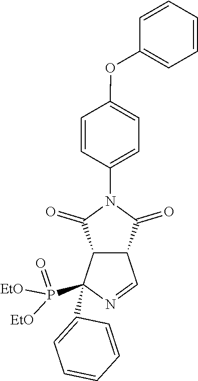 [SMILES: CCOP(=O)(OCC)[C@]1(c2ccccc2)N=C[C@H]2C(=O)N(c3ccc(Oc4ccccc4)cc3)C(=O)[C@H]21] | 6.96 ± 0.30 | 5.43 ± 0.21 | 34 |

### Parallel Artificial Membrane Permeation Assays-Blood-Brain Barrier (PAMPA-BBB)

To evaluate the brain penetration of the different compounds, a parallel artificial membrane permeation assay for blood-brain barrier was used, following the method described by Di et al. (cf. Table 2). The in vitro permeability (Pₑ) of fourteen commercial drugs through lipid extract of porcine brain membrane together with the test compounds were determined. Commercial drugs and assayed compounds were tested using a mixture of PBS:EtOH (70:30). Assay validation was made by comparing the experimental permeability with the reported values of the commercial drugs by bibliography and lineal correlation between experimental and reported permeability of the fourteen commercial drugs using the parallel artificial membrane permeation assay was evaluated (y=1.5481x−1.0128; r²=0,9405). From this equation and taking into account the limits established by Di et al. for BBB permeation, inventors have established the ranges of permeability as compounds of high BBB permeation (CNS+): Pe (10⁻⁶ cm s⁻¹)>5,179; compounds of low BBB permeation (CNS−): Pe (10⁻⁶ cm s⁻¹)<2,083 and compounds of uncertain BBB permeation (CNS+/−): 5,179>Pe (10⁻⁶ cm s⁻¹)>2,083. Table 2 shows permeability results from the different commercial and assayed compounds (three different experiments in triplicate) and predictive penetration in the CNS.

TABLE 2
Permeability (Pe 10−6 cm s⁻¹) in the PAMPA-BBB assay of 14
commercial drugs and tested compounds and predictive penetration
in the CNS. Bibliography values are taken from L. Di et al., “High
throughput artificial membrane permeability assay for blood-brain
barrier”, Eur. J. Med. Chem. 2003, vol. 38, pp. 223-232.

| | Bibliography | Experimental value ± SD | CNS |
|---|---|---|---|
| Compound | value | (n = 3) | Prediction |
| verapamil | 16.0 | 26.4 ± 0.5 | |
| testosterone | 17.0 | 24.9 ± 0.4 | |
| costicosterone | 5.1 | 6.7 ± 0.1 | |
| clonidine | 5.3 | 6.50 ± 0.05 | |
| ofloxacin | 0.8 | 0.99 ± 0.04 | |
| lomefloxacin | 0.0 | 0.70 ± 0.04 | |
| progesterone | 9.3 | 16.8 ± 0.3 | |
| promazine | 8.8 | 13.8 ± 0.3 | |
| imipramine | 13.0 | 12.3 ± 0.1 | |
| hidrocortisone | 1.9 | 1.40 ± 0.05 | |
| piroxicam | 2.5 | 1.90 ± 0.02 | |
| desipramine | 12.0 | 17.8 ± 0.1 | |
| cimetidine | 0.0 | 0.70 ± 0.03 | |
| norfloxacine | 0.1 | 0.90 ± 0.02 | |
| I-06 | | 9.7 ± 0.7 | CNS+ |
| I-07 | | 25.9 ± 0.6 | CNS+ |
| I-16 | | 7.8 ± 0.6 | CNS+ |
| I-22 | | 3.90 ± 0.15 | CNS+/− |
| I-25 | | 4.6 ± 0.7 | CNS+/− |
| I-26* | | 2.3 ± 0.04 | CNS+/− |
| I-28* | | 4.50 ± 0.25 | CNS+/− |
| I-29* | | >30 | CNS+ |
| I-30 | | 0.75 ± 0.13 | CNS− |
| I-32 | | 8.2 ± 0.3 | CNS+ |
| I-33 | | 6.2 ± 0.3 | CNS+ |
| I-34 | | 6.5 ± 0.02 | CNS+ |
| I-36 | | 7.2 ± 0.45 | CNS+ |
| I-37 | | 3.2 ± 0.2 | CNS+/− |
| I-38 | | 5.6 ± 0.15 | CNS+ |
| I-44 | | 8.9 ± 0.3 | CNS+ |
| I-45 | | 8.75 ± 0.5 | CNS+ |
| I-46 | | 6.9 ± 0.3 | CNS+ |
| I-47* | | 4.4 ± 0.4 | CNS+/− |
| I-49 | | 5.5 ± 0.03 | CNS+ |
| I-50* | | 1.4 ± 0.04 | CNS− |
| I-51 | | 6.2 ± 0.3 | CNS+ |
| I-52 | | 6.9 ± 0.2 | CNS+ |
| I-53* | | 3.7 ± 0.1 | CNS+/− |
| I-54 | | 3.7 ± 0.5 | CNS+/− |
| I-55 | | 6.3 ± 0.3 | CNS+ |
| I-56 | | 1.4 ± 0.06 | CNS− |
| I-57 | | 5.1 ± 0.01 | CNS+/− |

*= poor dissolution.
SD = Standard Deviation.

### Strains

SAMP8 is one of the non transgenic mouse model used in AD preclinical studies. It is an inbred strain with shortened lifespan and accelerated aging phenotype, with elevated levels of endogenous APP, but not plaques, and tau hyperphosphorylation. 5×FAD is an early-onset mouse transgenic model which overexpress mutant human APP(695) with the Swedish (K670N, M671L), Florida (1716V, and London (V7171) Familial Alzheimer's Disease (FAD) mutations along with human PS1 harboring two FAD mutations, M146L and L286V. Both transgenes are regulated by the mouse Thy1 promoter to drive overexpression in the brain. 5×FAD mice recapitulate major features of AD amyloid pathology and may be a useful model of intraneuronal Abeta-42 induced neurodegeneration and amyloid plaque formation.

Behavioral Test: In Vivo Model for Assessing the Efficacy of a Test Compound in Learning and Memory Impairment (Novel Object Recognition Test; NORT) (Cf. Tables 3-6)

Mice were placed in a 90°, two-arm, 25-cm-long, 20-cm-high, 5-cm-wide black maze. The walls could be lifted off for easy cleaning. Light intensity in the middle of the field was 30 lux. The objects to be discriminated were made of plastic and were chosen not to frighten the mice, and objects with parts that could be bitten were avoided. Before performing the test, the mice were individually habituated to the apparatus for 10 min during 3 days. On day 4, the animals were submitted to a 10-min acquisition trial (first trial), during which they were placed in the maze in the presence of two identical, novel objects (A+A or B+B) at the end of each arm. A 10-min retention trial (second trial) was carried out 2 h later. During this second trial, objects A and B were placed in the maze and the behavior of the mice was recorded with a camera. Time that the mice explored the new object (TN) and Time that the mice explored the Old object (TO) were measured. A Discrimination Index (DI) was defined as (TN−TO)/(TN+TO). In order to avoid object preference biases, A and B were counterbalanced so that one half of the animals in each experimental group were exposed first to A and then to B, whereas the remaining half saw B first and then A. The maze and the objects were cleaned with 70° ethanol after each test in order to eliminate olfactory cues. The learning & memory paradigm is based on spontaneous exploratory activity of rodents and does not involve rule learning or reinforcement. The object recognition paradigm has been shown to be sensitive to the effects of ageing and cholinergic dysfunction among others (cf. C. Scali et al., “Nerve growth factor increases extracellular acetylcholine levels in the parietal cortex and hippocampus of aged rats and restores object recognition”, Neurosci. Lett. 1994, vol. 170, pp 117-120; L. Bartolini et al., “Aniracetam restores object recognition impaired by age, scopolamine and nucleus basalis lesions”, Pharmacol. Biochem. Behav. 1996, vol. 53, pp. 277-283). This model has been adapted to mice and validated using pharmacological agents (cf. X. Bengoetxea et al., “Object recognition test for studying cognitive impairments in animal models of Alzheimer's disease”, Front. Biosci. (Schol. Ed.) 2015, vol. 7, pp 10-29). Evaluation of I-06 neuroprotective properties in both mice models by NORT, showed a reduced memory deficits in treated groups compared to the control, and in case of 5×FAD treated group recovered DI levels of the Wild Type (Wt) control group. Therefore, I-06 is able to improve cognitive capabilities in two murine models of neurodegeneration and brain disorders.

TABLE 3
Values of DI of NORT 2 h in female mice at 12 months control
SAMR1 (SAMR1-Ct), control SAMP8 (SAMP8-Ct), and SAMP8 treated
with I-06 (SAMP8-I-06). Data are observed mean ± Standard
Error of the Mean (SEM) (n = 8 for each group).

| NORT 2 h | | |
|---|---|---|
| Group | Number of mice | DI (−1, 1) |
| SAMR1-Ct | 8 | 0.37 ± .0.0427 |
| SAMP8-Ct | 7 | −0.023 ± 0.039**** |
| SAMP8-I-06 (5 mg/kg) | 7 | 0.33 ± 0.071^{##} |

****p < 0.0001 compared to SAMR1-Ct group.
^{##}p < 0.01 compared to SAMP8-Ct group.

TABLE 4
Values of DI of NORT 24 h in female mice at 12 months control
SAMR1 (SAMR1-Ct), control SAMP8 (SAMP8-Ct), and SAMP8 treated
with I-06 (SAMP8-I-06). (n = 8 for each group).

| NORT 24 h | | |
|---|---|---|
| Group | Number of mice | DI (−1, 1) |
| SAMR1-Ct | 8 | 0.295 ± 0.057 |
| SAMP8-Ct | 7 | −0.052 ± 0.091** |
| SAMP8 I-06 (5 mg/kg) | 7 | 0.273 ± 0.042^{##} |

**p < 0.01 compared to SAMR1-Ct group.
^{##}p < 0.01 compared to SAMP8-Ct group.

TABLE 5
Values of DI of NORT 2 h in female mice at 6 months control
Wt (Wt-Ct), control 5XFAD (5XFAD-Ct) and 5XFAD treated
with I-06 (5XFAD-I-06). (n = 8 for each group).

| NORT 2 h | | |
|---|---|---|
| Group | Number of mice | DI (−1, 1) |
| Wt-Ct | 14 | 0.12 ± 0.33 |
| 5xFAD-Ct | 16 | −0.032 ± 0.067* |
| 5xFAD I-06 (5 mg/kg) | 18 | 0.199 ± 0.053^{#} |

*p < 0.05 compared to Wt-Ct group.
^{#}p < 0.05 compared to 5xFAD-Ct group.

TABLE 6
Values of DI of NORT 24 h in female mice at 6 months control
Wt (Wt-Ct), control 5XFAD (5XFAD-Ct) and 5XFAD treated
with I-06 (5XFAD-I-06). (n = 8 for each group).

| NORT 24 h | | |
|---|---|---|
| Group | Number of mice | DI (−1, 1) |
| Wt-Ct | 14 | 0.211 ± 0.054 |
| 5xFAD-Ct | 16 | −0.060 ± 0.076* |
| 5xFAD I-06 (5 mg/kg) | 18 | 0.229 ± 0.051^{#} |

*p < 0.05 compared to Wt-Ct group.
^{#}p < 0.05 compared to 5xFAD-Ct group.

### Molecular Analysis: RNA Extraction and Gene Expression Determination for Inflammatory Markers and Beta Amyloid Processing

Total RNA isolation was carried out by means of Trizol reagent following manufacturer's instructions. RNA content in the samples was measured at 260 nm, and sample purity was determined by the A260/280 ratio in a NanoDrop™ ND-1000 (Thermo Scientific). Samples were also tested in an Agilent 2100B Bioanalyzer (Agilent Technologies) to determine the RNA integrity number. Reverse Transcription-Polymerase Chain Reaction (RT-PCR) was performed as follows: 2 μg of messenger RNA (mRNA) was reverse-transcribed using the High Capacity complementary DNA (cDNA) Reverse Transcription Kit (Applied Biosystems). Normalization of expression levels was performed with actin. SYBER Green, real-time PCR was performed in the Step One Plus Detection System (Applied Biosystems) employing the SYBR Green PCR Master Mix. Each reaction mixture contained 7.5 μL of cDNA, whose concentration was 2 μg, 0.75 μL of each primer (whose concentration was 100 nM), and 7.5 μL of SYBR Green PCR Master Mix (2×). Data were analyzed utilizing the comparative Cycle threshold (Ct) method (Fold Change), where the actin transcript level was used to normalize differences in sample loading and preparation. Each sample (n=4-5) was analyzed in triplicate, and results represented the n-fold difference of transcript levels among different samples.

Effect of I-06 on Inflammatory Markers (Cf. Tables 7-8)

Molecular evaluation of inflammatory markers in brain by gene expression determination shows that I-06 reduced two markers of neuroinflammation such as II-6 and Cxcl10, both in SAMP8 and 5×FAD.

TABLE 7
Values of gene expression of Inflammatory Markers for II-6
and Cxcl10 in female mice at 12 months control SAMR1 (SAMR1-
Ct), control SAMP8 (SAMP8-Ct), and SAMP8 treated with I-06
(SAMP8-I-06). Gene expression levels were determined by real-
time PCR. Mean ± SEM from five independent experiments
performed in triplicate are represented (n = 4 for each group).

| Group | Gene Name | Fold Change |
|---|---|---|
| SAMR1-Ct | II-6 | 0.486 ± 0.0228 |
| | Cxcl10 | 0.563 ± 0.125 |
| SAMP8-Ct | II-6 | 1.006 ± 0.078**** |
| | Cxcl10 | 1.02 ± 0.158* |
| SAMP8-I-06 (5 mg/kg) | II-6 | 0.701 ± 0.070^{#} |
| | Cxcl10 | 0.660 ± 0.012 |

*p < 0.05 compared to SAMR1-Ct group.
****p < 0.0001 compared to SAMR1-Ct group.
^{#}p < 0.05 compared to SAMP8-Ct group.

TABLE 8
Values of gene expression of Inflammatory Markers for II-6
and Cxcl10 in female mice at 6 months control Wt (Wt-Ct),
control 5XFAD (5XFAD-Ct), and 5XFAD treated with I-06 (5XFAD-
I-06). Gene expression levels were determined by real-time
PCR. Mean ± SEM from five independent experiments performed
in triplicate are represented (n = 4 for each group).

| Group | Gene Name | Fold Change |
|---|---|---|
| Wt-Ct | II-6 | 1.002 ± 0.036 |
| | Cxcl10 | 1.193 ± 0.413 |
| 5xFAD-Ct | II-6 | 1.2474 ± 0.101* |
| | Cxcl10 | 3.541 ± 1.153 |
| 5xFAD-I-06 (5 mg/kg) | II-6 | 1.35 ± 0.136 |
| | Cxcl10 | 2.07 ± 0.647 |

*p < 0.05 compared to Wt-Ct group.

qPCR: APP Processing and Degradation Markers (Cf. Table 9)

With the aim to describe the amyloid precursor protein (APP) processing and Abeta degradation, inventors evaluated the gene expression of Adam10, Neprilysin (Nep) and Insuline Degrading Enzyme (Ide). Significantly increases in gene expression of Adam10 and Ide (two enzymes related with non-amiloidogenic processing of APP) in SAMP8 treated group compared to SAMP8-Ct group were obtained.

TABLE 9
Values of gene expression of APP processing and degradation
Markers for Adam10, Nep and Ide in female mice at 12 months
control SAMR1 (SAMR1-Ct), control SAMP8 (SAMP8-Ct), and SAMP8
treated with I-06 (SAMP8-I-06). Gene expression levels were
determined by real-time PCR. Results indicated improved processing
pathway for APP through a reduction in beta amyloid production.
Mean ± SEM from five independent experiments performed
in triplicate are represented (n = 4 for each group).

| Group | Gene Name | Fold Change |
|---|---|---|
| SAMR1-Ct | Adam10 | 1.467 ± 0.198 |
| | Nep | 1.614 ± 0.057 |
| | Ide | 1.263 ± 0.036 |
| SAMP8-Ct | Adam10 | 1.044 ± 0.209 |
| | Nep | 1.035 ± 0.269* |
| | Ide | 1.021 ± 0.114 |
| SAMP8-I-06 (5 mg/kg) | Adam10 | 1.88 ± 0.222^{#} |
| | Nep | 1.772 ± 0.226 |
| | Ide | 1.3436 ± 0.074^{#} |

*p < 0.05 compared to SAMR1-Ct group.
^{#}p < 0.05 compared to SAMP8-Ct group.

### CITATION LIST

### Non Patent Literature

S. Abas et al., “Easy access to (2-imidazolin-4-yl)phosphonates by a microwave assisted multicomponent reaction”, Tetrahedron 2015, vol. 71, pp. 2872-2881.
S. Abas et al., “Neuroprotective Effects of a Structurally New Family of High Affinity Imidazoline I₂ Receptor Ligands”, ACS Chemical Neuroscience 2017, vol. 8, pp. 737-742.
C. Arroniz et al., “First diastereoselective [3+2] cycloaddition reaction of diethyl isocyanomethylphosphonate and maleimides”, Org. Biomol. Chem. 2013, vol. 11, pp. 1640-1649.
L. Bartolini et al., “Aniracetam restores object recognition impaired by age, scopolamine and nucleus basalis lesions”, Pharmacol. Biochem. Behav. 1996, vol. 53, pp. 277-283.
X. Bengoetxea et al., “Object recognition test for studying cognitive impairments in animal models of Alzheimer's disease”, Front. Biosci. (Schol. Ed.) 2015, vol. 7, pp 10-29.
L. Di et al., “High throughput artificial membrane permeability assay for blood-brain barrier”, Eur. J. Med. Chem. 2003, vol. 38, pp. 223-232.
J.-X. Li, “Imidazoline I₂ receptors: An update”, Pharmacology & Therapeutics 2017. vol. 178, pp. 48-56.
J.-X. Li et al., “Imidazoline I₂ receptors: Target for new analgesics?”, European Journal of Pharmacology 2011, vol. 658, pp. 49-56.
J. Rachon et al., “Syntheses with α-metalated isocyanides. XLVIII. Phosphorus analogs of amino acids and peptides. V. Synthesis of 1-aminoalkylphosphonic acids by alkylation of α-metalated diethyl isocyanomethylphosphonate”, Liebigs Ann. Chem. 1981, pp. 709-718.
C. Scali et al., “Nerve growth factor increases extracellular acetylcholine levels in the parietal cortex and hippocampus of aged rats and re-stores object recognition”, Neurosci. Lett. 1994, vol. 170, pp 117-120.
M. Sieńczyk, “Synthesis of isocyanide derivatives of α-aminoalkylphosphonate diphenyl esters”, Tetrahedron Lett. 2006, vol. 47, pp. 4209-4211.
S. Sobhani et al., “Molecular Iodine: An efficient catalyst for the one-pot synthesis of primary 1-aminophosphonates”, J. Iran. Chem. Soc. 2010, vol. 7, pp. 227-236.
M. Sortino et al. “Antifungal, cytotoxic and SAR studies of a series of N-alkyl, N-aryl and N-alkylphenyl-1,4-pyrrolediones and related compounds”, Bioorg. Med. Chem. 2011, vol. 19, pp. 2823-2834.

1. A compound selected from the group consisting of the enantiomer of formula I, its mirror-image enantiomer, and a mixture of both enantiomers, wherein:
R₁ is ethyl or phenyl;
R₂ is methyl, phenyl, benzyl, monosubstituted phenyl, or monosubstituted benzyl, with a substituent being F, Cl, Br, (C₁-C₃)-alkyl, or (C₁-C₃)-alkyloxy; and
R₃ is a radical selected from the group consisting of (C₁-C₆)-alkyl, (C₁-C₆)-cycloalkyl, —[CH₂]ₙ-phenyl, —[CH₂]ₙ-1-naphtyl, —[CH₂]ₙ-2-naphtyl, and —[CH₂]ₙ-[substituted phenyl]; wherein [substituted phenyl] is a phenyl radical with one, two or three substituents which are radicals independently selected from the group consisting of F, Cl, Br, (C₁-C₃)-alkyl, (C₁-C₃)-alkyloxy, phenyl, phenoxy, —CF₃, —OCF₃, nitro, —CN, —CO—(C₁-C₃)-alkyl, and benzoyl; and n is an integer between 0 and 4.

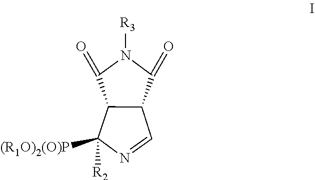

2. The compound according to claim 1, which is a mixture of both enantiomers.

3. The compound according to claim 2, wherein the mixture is a racemic mixture.

4. The compound according to claim 1, wherein the substituted phenyl is a phenyl radical with one or two substituents.

5. The compound according to claim 1, wherein n is an integer between 0 and 2.

6. The compound according to claim 1, wherein R₃ is selected from the group consisting of methyl, ethyl, n-propyl, tert-butyl, cyclohexyl, phenyl, 1-naphtyl, 4-methylphenyl, 4-methoxyphenyl, 4-phenoxyphenyl, 3-(trifluoromethyl)phenyl, 4-(trifluoromethyl)phenyl, 4-fluorophenyl, 2-, 3- and 4-chlorophenyl, 4-bromophenyl, 3- and 4-nitrophenyl, 3,4- and 3,5-dichlorophenyl, 3-chloro-4-fluorophenyl, 2-methyl-5-nitrophenyl, (1,1′-biphenyl)-4-yl, benzyl, phenethyl and 4-fluorophenethyl.

7. The compound according to claim 1, wherein R₁ is ethyl.

8. The compound according to claim 1, wherein R₂ is phenyl.

9. The compound according to claim 3, which is a racemic mixture selected from:
diethyl (1RS,3aSR,6aSR)-5-(3-chloro-4-fluorophenyl)-4,6-dioxo-1-phenyl-1,3a,4,5,6,6a-hexahydropyrrolo[3,4-c]pyrrole-1-phosphonate (I-06);
diethyl (1RS,3aRS,6aRS)-1,5-dimethyl-4,6-dioxo-1,3a,4,5,6,6a-hexahydropyrrolo[3,4-c]pyrrole-1-phosphonate (I-25);
diethyl (1RS,3aRS,6aRS)-1-methyl-4,6-dioxo-5-phenyl-1,3a,4,5,6,6a-hexahydropyrrolo[3,4-c]pyrrole-1-phosphonate (I-31);
diethyl (1RS,3aSR,6aSR)-5-methyl-4,6-dioxo-1-phenyl-1,3a,4,5,6,6a-hexahydropyrrolo[3,4-c]pyrrole-1-phosphonate (I-30);
diethyl (1RS,3aSR,6aSR)-5-cyclohexyl-4,6-dioxo-1-phenyl-1,3a,4,5,6,6a-hexahydropyrrolo[3,4-c]pyrrole-1-phosphonate (I-07);
diethyl (1RS,3aSR,6aSR)-5-benzyl-4,6-dioxo-1-phenyl-1,3a,4,5,6,6a-hexahydropyrrolo[3,4-c]pyrrole-1-phosphonate (I-29);
diethyl (1RS,3aSR,6aSR)-5-(3-nitrophenyl)-4,6-dioxo-1-phenyl-1,3a,4,5,6,6a-hexahydropyrrolo[3,4-c]pyrrole-1-phosphonate (I-28);
diethyl (1RS,3aSR,6aSR)-4,6-dioxo-1,5-diphenyl-1,3a,4,5,6,6a-hexahydropyrrolo[3,4-c]pyrrole-1-phosphonate (I-16);
diethyl (1RS,3aSR,6aSR)-5-(4-methoxyphenyl)-4,6-dioxo-1-phenyl-1,3a,4,5,6,6a-hexahydropyrrolo[3,4-c]pyrrole-1-phosphonate (I-22);
diphenyl (1RS,3aSR,6aSR)-4,6-dioxo-1,5-diphenyl-1,3a,4,5,6,6a-hexahydropyrrolo[3,4-c]pyrrole-1-phosphonate (I-26);
diethyl (1RS,3aSR,6aSR)-4,6-dioxo-5-phenethyl-1-phenyl-1,3a,4,5,6,6a-hexahydropyrrolo[3,4-c]pyrrole-1-phosphonate (I-33);
diethyl (1RS,3aSR,6aSR)-5-(1,1′-biphenyl)-4-yl-4,6-dioxo-1-phenyl-1,3a,4,5,6,6a-hexahydropyrrolo[3,4-c]pyrrole-1-phosphonate (I-32);
diethyl (1RS,3aSR,6aSR)-4,6-dioxo-5-(4-phenoxyphenyl)-1-phenyl-1,3a,4,5,6,6a-hexahydropyrrolo[3,4-c]pyrrole-1-phosphonate (I-34)
diethyl (1RS,3aSR,6aSR)-5-(4-fluorophenethyl)-4,6-dioxo-1-phenyl-1,3a,4,5,6,6a-hexahydropyrrolo[3,4-c]pyrrole-1-phosphonate (I-36)
diethyl (1RS,3aSR,6aSR)-5-(4-fluorophenyl)-4,6-dioxo-1-phenyl-1,3a,4,5,6,6a-hexahydropyrrolo[3,4-c]pyrrole-1-phosphonate (I-37)
diethyl (1RS,3aSR,6aSR)-4,6-dioxo-1-phenyl-5-[4-(trifluoromethyl)phenyl]-1,3a,4,5,6,6a-hexahydropyrrolo[3,4-c]pyrrole-1-phosphonate (I-38)
diethyl (1RS,3aSR,6aSR)-5-(3,4-dichlorophenyl)-4,6-dioxo-1-phenyl-1,3a,4,5,6,6a-hexahydropyrrolo[3,4-c]pyrrole-1-phosphonate (I-44);
diethyl (1RS,3aSR,6aSR)-4,6-dioxo-1-phenyl-5-(p-tolyl)-1,3a,4,5,6,6a-hexahydropyrrolo[3,4-c]pyrrole-1-phosphonate (I-45);
diethyl (1RS,3aSR,6aSR)-1-benzyl-4,6-dioxo-5-phenyl-1,3a,4,5,6,6a-hexahydropyrrolo[3,4-c]pyrrole-1-phosphonate (I-46);
diethyl (1RS,3aSR,6aSR)-5-(4-bromophenyl)-4,6-dioxo-1-phenyl-1,3a,4,5,6,6a-hexahydropyrrolo[3,4-c]pyrrole-1-phosphonate (I-47);
diethyl (1RS,3aSR,6aSR)-5-(4-chlorophenyl)-4,6-dioxo-1-phenyl-1,3a,4,5,6,6a-hexahydropyrrolo[3,4-c]pyrrole-1-phosphonate (I-49);
diethyl (1RS,3aSR,6aSR)-5-(2-methyl-5-nitrophenyl)-4,6-dioxo-1-phenyl-1,3a,4,5,6,6a-hexahydropyrrolo[3,4-c]pyrrole-1-phosphonate (I-50);
diethyl (1RS,3aSR,6aSR)-4,6-dioxo-1-phenyl-5-propyl-1,3a,4,5,6,6a-hexahydropyrrolo[3,4-c]pyrrole-1-phosphonate (I-51);
diethyl (1RS,3aSR,6aSR)-4,6-dioxo-1-phenyl-5-[3-(trifluoromethyl)phenyl]-1,3a,4,5,6,6a-hexahydropyrrolo[3,4-c]pyrrole-1-phosphonate (I-52);
diethyl (1RS,3aSR,6aSR)-5-(3-chlorophenyl)-4,6-dioxo-1-phenyl-1,3a,4,5,6,6a-hexahydropyrrolo[3,4-c]pyrrole-1-phosphonate (I-53);
diethyl (1RS,3aSR,6aSR)-5-ethyl-4,6-dioxo-1-phenyl-1,3a,4,5,6,6a-hexahydropyrrolo[3,4-c]pyrrole-1-phosphonate (I-54);
diethyl (1RS,3aSR,6aSR)-5-(tert-butyl)-4,6-dioxo-1-phenyl-1,3a,4,5,6,6a-hexahydropyrrolo[3,4-c]pyrrole-1-phosphonate (I-55);
diethyl (1RS,3aSR,6aSR)-5-(naphth-1-yl)-4,6-dioxo-1-phenyl-1,3a,4,5,6,6a-hexahydropyrrolo[3,4-c]pyrrole-1-phosphonate (I-56);
diethyl (1RS,3aSR,6aSR)-1-benzyl-5-cyclohexyl-4,6-dioxo-1,3a,4,5,6,6a-hexahydropyrrolo[3,4-c]pyrrole-1-phosphonate (I-57);
diethyl (1RS,3aSR,6aSR)-5-(3,5-dichlorophenyl)-4,6-dioxo-1-phenyl-1,3a,4,5,6,6a-hexahydropyrrolo[3,4-c]pyrrole-1-phosphonate (I-58);
diethyl (1RS,3aSR,6aSR)-5-(4-nitrophenyl)-4,6-dioxo-1-phenyl-1,3a,4,5,6,6a-hexahydropyrrolo[3,4-c]pyrrole-1-phosphonate (I-59);
diethyl (1RS,3aSR,6aSR)-5-(3-chloro-4-fluorophenyl)-1-(4-fluorophenyl)-4,6-dioxo-1,3a,4,5,6,6a-hexahydropyrrolo[3,4-c]pyrrol-1-phosphonate (I-62);
diethyl (1RS,3aSR,6aSR)-5-(3-chloro-4-fluorophenyl)-1-(4-methoxyphenyl)-4,6-dioxo-1,3a,4,5,6,6a-hexahydropyrrolo[3,4-c]pyrrole-1-phosphonate (I-64);
diethyl (1RS,3aSR,6aSR)-1-(4-fluorophenyl)-4,6-dioxo-5-phenyl-1,3a,4,5,6,6a-hexahydropyrrolo[3,4-c]pyrrole-1-phosphonate (I-65);
diethyl (1RS,3aSR,6aSR)-1-(4-methoxyphenyl)-4,6-dioxo-5-phenyl-1,3a,4,5,6,6a-hexahydropyrrolo[3,4-c]pyrrole-1-phosphonate (I-66);
diethyl (1RS,3aSR,6aSR)-5-cyclohexyl-1-(4-fluorophenyl)-4,6-dioxo-1,3a,4,5,6,6a-hexahydropyrrolo[3,4-c]pyrrole-1-phosphonate (I-67);
diethyl (1RS,3aSR,6aSR)-5-cyclohexyl-1-(4-methoxyphenyl)-4,6-dioxo-1,3a,4,5,6,6a-hexahydropyrrolo[3,4-c]pyrrole-1-phosphonate (I-68); and
diethyl (1RS,3aSR,6aSR)-5-(2-chlorophenyl)-4,6-dioxo-1-phenyl-1,3a,4,5,6,6a-hexahydropyrrolo[3,4-c]pyrrole-1-phosphonate (I-69).

10. Diethyl (1RS,3aSR,6aSR)-5-(3-chloro-4-fluorophenyl)-4,6-dioxo-1-phenyl-1,3a,4,5,6,6a-hexahydropyrrolo[3,4-c]pyrrole-1-phosphonate (I-06).

11. (canceled)

12. A method of prevention or treatment of a brain disorder in an animal, including a human, said method comprising administering the compound of claim 1 to said animal.

13. The method according to claim 12, wherein the brain disorder is a neurodegenerative disorder.

14. The method according to claim 13, wherein the neurodegenerative disorder is Alzheimer's disease.
